(19) United States
(12) Patent Application Publication
(10) Pub. No.: US 2007/0004989 A1
(43) Pub. Date: Jan. 4, 2007

(54) Device for transdermal sampling

(76) Inventor: Parvinder Dhillon, Fremont, CA (US)

Correspondence Address: GluQuest, Inc., 525 Ondina Drive, Fremont, CA, 94539, US

(21) Appl. No.: 11/171,581

(22) Filed: Jun. 29, 2005

(51) Int. Cl. A61B 5/00

(52) U.S. Cl. 600/583

(57) ABSTRACT

Transdermal agent sampling devices are described which combine arrays of puncturing elements, do not require the use of pumps, and in which the sensing means for detecting the agent is directly proximal to, or comprised within, the array of puncturing elements. An array design that improves the flow of fluid from the skin to the sensor, allowing efficient utilization of the extracted fluid is also described. Devices that are suitable for use in a patch for agent monitoring, in that they are smaller and cheaper to manufacture, as well as being lighter, less obtrusive, and less irritating to the user are also described.

### TECHNICAL FIELD

The present invention relates to devices for interstitial fluid sampling, in particular to devices for glucose monitoring.

### BACKGROUND

Standard commercially available glucose monitoring devices utilize fingerstick or alternate site testing. What these methods have in common is that in almost every case a sample of blood must be obtained using a separate lancing device, and that sample is then applied to the test strip and a reading is obtained. The major drawbacks of these devices are that to get a glucose reading the user must undergo a considerable hassle with the meter and a lancet device, obtain an adequate blood sample, apply it to the test strip, and subsequently dispose of used strip, lancet, packaging, and so on. This is not to mention the pain, tenderness and callousing that occurs with repeated fingersticking. Diabetics must regularly self-test themselves several times per day. Each test requires a separate lancing, each of which involves an instance of pain for the user. Another problem associated with some conventional lancing devices is that the lacerations produced by the lances are larger than necessary and consequently take a greater time to heal. The greater the amount of time for the wound to heal translates into a longer period of time in which the wound is susceptible to infection.

Completely non-invasive methods for glucose monitoring have been proposed. In these proposed products, the glucose levels are to be obtained without extracting any fluids from the body. Instead, light, sound, radio or other waveforms are refracted, scattered, or absorbed within the body and those effects are measured and converted into glucose concentrations (see, for example, U.S. Pat. No. 6,505,059). These methods typically detect only changes in glucose concentration, not absolute values, thus requiring frequent references back to baseline (i.e., fingersticks). Since no fluids are extracted, the readings must be made through the skin or some other non-invasive portal to body fluids, making such readings susceptible to changes in temperature, perspiration, skin pigmentation, and other potential influences. Finally, the task of getting a sufficiently robust “signal” and separating it from the vast background of “noise” remains extremely challenging.

Somewhat more progress has been made on minimally invasive glucose monitoring devices. A common feature of these devices is that they monitor glucose levels in interstitial fluid instead of blood. Interstitial fluid is the substantially clear, substantially colorless fluid found in the human body that occupies the space between the cells of the human body. Diagnostic tests that can be run with samples of interstitial fluid include, but are not limited to, glucose, creatinine, BUN, uric acid, magnesium, chloride, potassium, lactate, sodium, oxygen, carbon dioxide, triglyceride, and cholesterol.

It is much more difficult to obtain a sample of interstitial fluid from the body of a patient than it is to obtain a sample of blood from the body of a patient. Blood is pumped under pressure through blood vessels by the heart. Consequently, a cut in a blood vessel will naturally lead to blood flowing out of the cut because the blood is flowing under pressure. Interstitial fluid, which is not pumped through vessels in the body, is under a slight negative pressure, or suction. Moreover, the amount of interstitial fluid that can be obtained from a patient is small because this fluid only occupies the space between the cells of the human body.

Several methods have been employed to obtain access to interstitial fluid for diagnostic tests, including glucose monitoring. These methods include, but are not limited to, microdialysis, heat poration, open flow microperfusion, ultrafiltration, subcutaneous implantation of a sensor, needle extraction, reverse iontophoresis, suction effusion, and ultrasound.

Currently available devices include the GLUCOWATCH BIOGRAPHER by Cygnus and the CGMS GUARDIAN by Medtronic; awaiting FDA action is the FREESTYLE NAVIGATOR by TheraSense (Abbott). (See Tierney, M.J., IDV Technology, May 2003, p. 51). These devices have drawbacks in that interstitial fluid must be obtained invasively to test for glucose (using either a collection needle or iontophoresis). Proposed alternatives to the needle require the use of lasers or heat (see, for example, WO 97/07734 and U.S. Pat. No. 6,508,785) to create a hole in the skin, which is inconvenient, expensive, or undesirable for repeated use. The reverse iontophoresis method used in the Cygnus device causes skin irritation, and is also subject to an initial time delay for retrieval of sufficient fluid for sampling. The implantable sensor utilized by Medtronic is difficult to calibrate because it is located inside the body. Furthermore, the sensor is subject to the motion of the body as well as to attacks by the body's immune system. A ftrther drawback to these devices is that they are not intended as a replacement for fingerstick testing of glucose, but rather as an adjunct to it. The devices must be calibrated periodically to glucose measurements taken by fingerstick methods.

Methods and devices are known in the art for increasing interstitial fluid flow by mechanically puncturing the skin using arrays of skin puncturing elements such as microneedles or microblades. (See, for example, U.S. Pat. No. 3,964,482, WO 98/00193, WO 99/64580, WO 00/74763, WO 96/37256, U.S. Pat. No. 6,219,574.) Skin consists of multiple layers, of which the stratum corneum layer is the outermost layer, followed by a viable epidermal layer, and fmally a dermal tissue layer. The thin layer of stratum corneum is the major barrier for agent passage through the skin. Microneedles or microblades are used to create holes or slits in the stratum corneum for agent sampling. When the needles or blades do not penetrate down to the nerve endings, there is no pain or bleeding.

Due to the difficulties in extracting interstitial fluid, known devices typically couple the microneedle or microblade array to another extraction method, such as electrophoresis, ultrasound, or negative pressure (suction) provided by a pump. These additions add to the bulk or complexity of the device, or cause irritation of the skin. Microblade devices utilizing passive diffusion methods have been described (for example, in U.S. Pat. No. 6,219,574), but in these devices the system for sensing the glucose or other agent is located above an absorbent pad or fluid reservoir, requiring that sufficient fluid be extracted to fill the fluid reservoir before the agent can be sensed. A further issue is that after puncturing the skin, the fluid must be able to penetrate through the base of the array, typically through holes in the array base, in order to reach the sensor. As the skin can conform around the base of the array, fluid flow from the puncture sites to the holes in the array base can become blocked.

There is a need in the art for devices which permit continual, unlimited reading, minimally invasive monitoring of glucose or other agents. Such devices would also preferably be compact, non-irritating, and easy to use, so as to permit wear for extended periods (i.e., 1-3 days).

It is an object and advantage of the invention to provide transdermal agent sampling devices which combine arrays of puncturing elements with collectors which provide means for evaporation of sampled fluid from the device, generating an increased motive force for passive diffusion to draw out the interstitial fluid. Thus the devices of the invention do not require pumps, which add to the bulk of the device, or electrophoretic or ultrasound methods which can cause skin irritation. It is a further object and advantage of the invention to provide devices in which the sensing means for detecting the agent is directly proximal to, or comprised within, the array of puncturing elements, thus requiring smaller sample sizes and allowing for more rapid sensing, as little fluid is wasted, and it is not necessary to fill a fluid reservoir before agent detection can occur. It is a further object and advantage of the invention to provide an array design that improves the flow of fluid from the skin to the sensor, allowing efficient utilization of the extracted fluid. Since very little fluid sample is required for the sensor to measure the agent, the array of puncturing elements can have a very small area, resulting in the disruption of a smaller skin area and therefore reduced skin irritation effects. It is a further object and advantage of the invention to provide devices that are suitable for use in a patch for agent monitoring, in that they are smaller and cheaper to manufacture, as well as being lighter, less obtrusive, and less irritating to the user. Still further objects and advantages will become apparent to one of ordinary skill in the art from a consideration of the ensuing description and drawings.

### SUMMARY

In accordance with the invention, a device for sampling of agents in interstitial fluid comprises a base having a lower side and an upper side; a plurality of puncturing elements extending from the lower side of the base; a plurality of holes extending from the lower side of the base to the upper side of the base, the holes configured for permitting a liquid to move therethrough, a network of channels configured in the lower side of the base to interconnect the holes; and one or more protrusions extending from the lower side of the base, the protrusions of sufficient height and width to allow fluid to flow under the base while still permitting the puncturing elements to penetrate through the stratum comeum of a subject. Embodiments of the device may further comprise an agent sensing element such as a bioelectrochemical sensor, wherein the agent sensing element is contiguous with the upper side of the base, or comprised within the puncturing elements. This configuration allows for more rapid agent detection, and requires smaller sample sizes, as little fluid is wasted, and it is not necessary to fill a fluid reservoir before agent detection can occur.

The invention further provides a collector that may be used in combination with the array of puncturing elements or with other skin piercing arrays. The collector comprises an absorbent membrane disposed above the array and agent sensing element to absorb the interstitial fluid. The collector further comprises means for increasing the rate of evaporation of the interstitial fluid, for example slits in a casing which houses the collector membrane, and/or a heating element.

The invention contemplates the use of the disclosed array of puncturing elements and the disclosed collector as elements of an integrated agent sampling device, or for use independently in combination with other skin puncturing devices or collectors known in the art. The invention further contemplates the use of the disclosed skin puncturing and collector devices together with additional components as components of a “smart patch” for monitoring and/or regulating levels of an agent, for example as a patch for monitoring and/or regulating glucose levels in diabetic patients.

### BRIEF DESCRIPTION OF THE FIGURES

FIG. 1 is an enlarged diagrammatic cross-sectional view of a skin piercing array in accordance with one embodiment of the present invention.

FIG. 2 is an enlarged perspective view of the skin proximal side of the array.

FIGS. 3A-G show various possible shapes for the puncturing elements of the sampling system; FIG. 3H shows an embodiment of a puncturing element with surface texturing; FIG. 3I (shows a cross-section of the element of FIG. 3H.

FIG. 4 shows various possible shapes for the “bumps” of the skin piercing array.

FIG. 5 shows various possible shapes for the channels of the skin piercing array.

FIG. 6 shows various possible configurations for the holes in the skin piercing array.

FIGS. 7A and 7B show cross-sectional views of alternative embodiments of the skin piercing array of the invention.

FIG. 8 is a diagrammatic cross-sectional view of a collector in accordance with one embodiment of the present invention.

### DESCRIPTION

The term “sampling” is used broadly herein to include withdrawal of or monitoring the presence or amount of an agent. The term “agent” broadly includes substances such as glucose, body electrolytes, alcohol, illicit drugs, licit substances, pharmaceuticals, blood gases, etc. that can be sampled through the skin.

Preferred Embodiments:

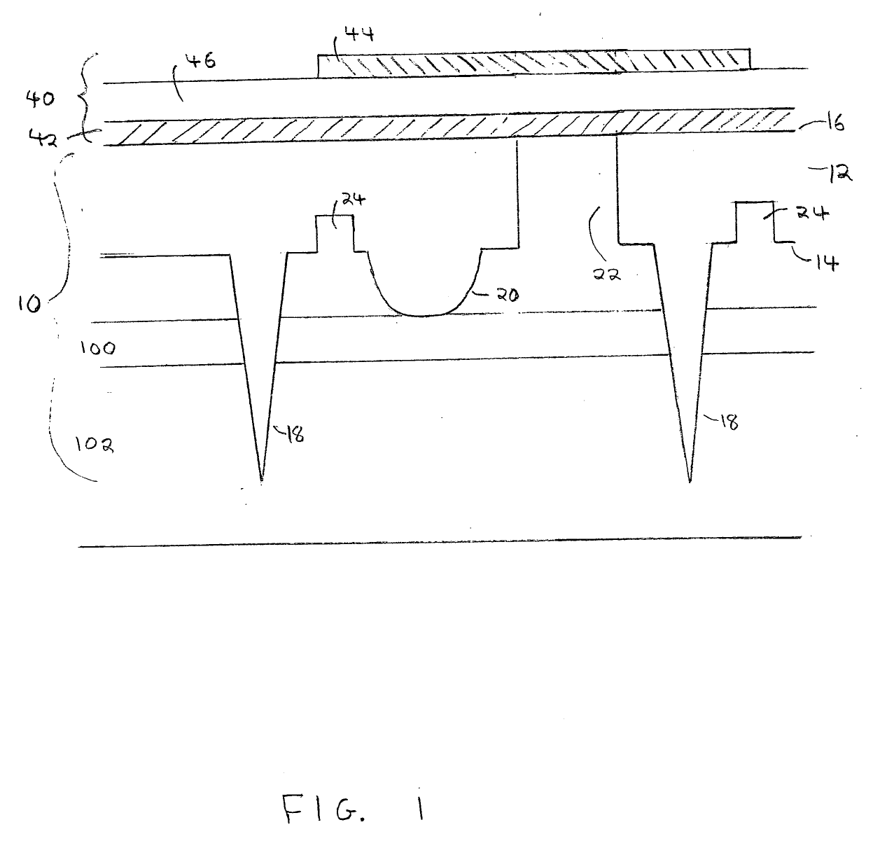

One embodiment of the transdermal agent sampling device of the present invention is illustrated in FIG. 1. The device comprises a base (12) with an upper side (16) and a lower side (14). A plurality of skin puncturing elements (18) project at an angle from the lower side (14) of the base. The puncturing elements (18) are sized and shaped to penetrate the stratum comeum (100) of the skin when pressure is applied to the device, but do not penetrate the skin sufficiently to contact the subject's nerve endings. In the embodiment of the invention shown in FIG. 1, the puncturing elements (18) are microneedles. The microneedles are preferably from about 50 microns to about 500 microns in length, dependent upon the skin type of the intended subject. The cross section of the needles is preferably from about 50 microns to about 500 microns in width, dependent upon the process and substrate used to produce them.

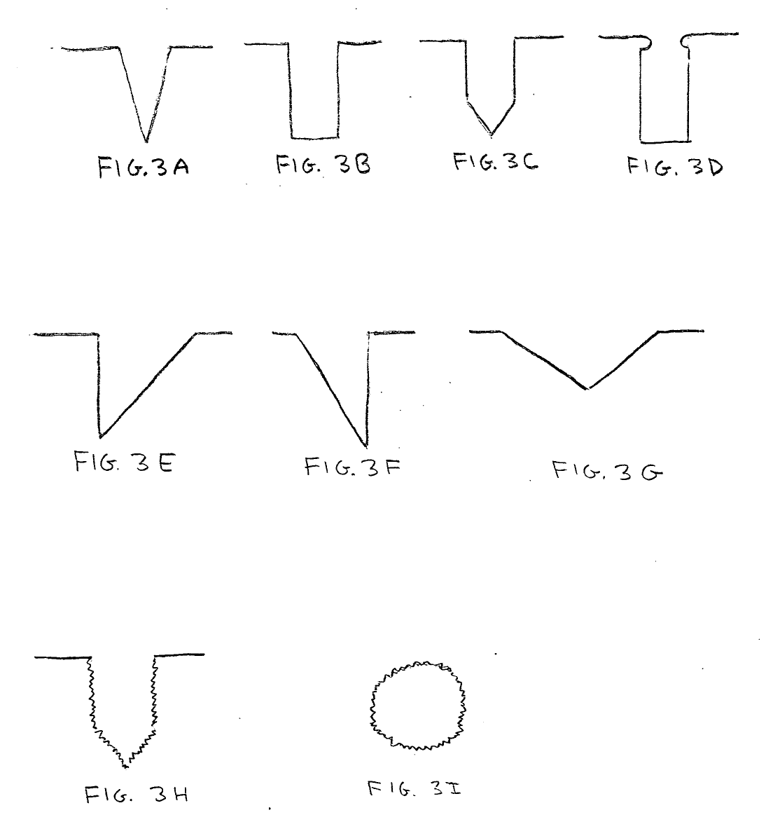

The angular relationship between the puncturing elements (18) and the corresponding device base surface (14) is preferably perpendicular, although an exact right angle of 90 degrees is not required. In one embodiment, the puncturing elements (18) are microneedles with a slight undercut at the base of each microneedle, as depicted in FIG. 3D.

Although the puncturing elements are depicted as microneedles, the puncturing elements are not limited to elements having a cylindrical needle shape. The shape of the puncturing elements may vary depending upon the substrate material, the fabrication process, the required useful life of the puncturing elements, the methods in which they will be used, cost constraints and other parameters. Illustrative examples of possible shapes for the puncturing elements are shown in FIGS. 3A-3G. The shape of the puncturing elements may include any other shape suitable for penetrating the stratum comeum of the epidermis without penetrating the skin sufficiently to contact the subject's nerve endings, including but not limited to microneedles with beveled ends or other asymmetric tips as disclosed in U.S. Pat. No. 6,558,361, microneedles with triangular or star-shaped tips as in U.S. Pat. No. 6,652,478, wedge shaped elements as disclosed in WO 98/00193, and microblades as disclosed in U.S. Pat. No. 6,219,574.

The density of puncturing elements can have a wide range depending on the dimensions of the puncturing elements (length, width, aspect ratio and shape), the fabrication methods, and the substrate material, but is preferably from about 2 to about 20 puncturing elements per square millimeter.

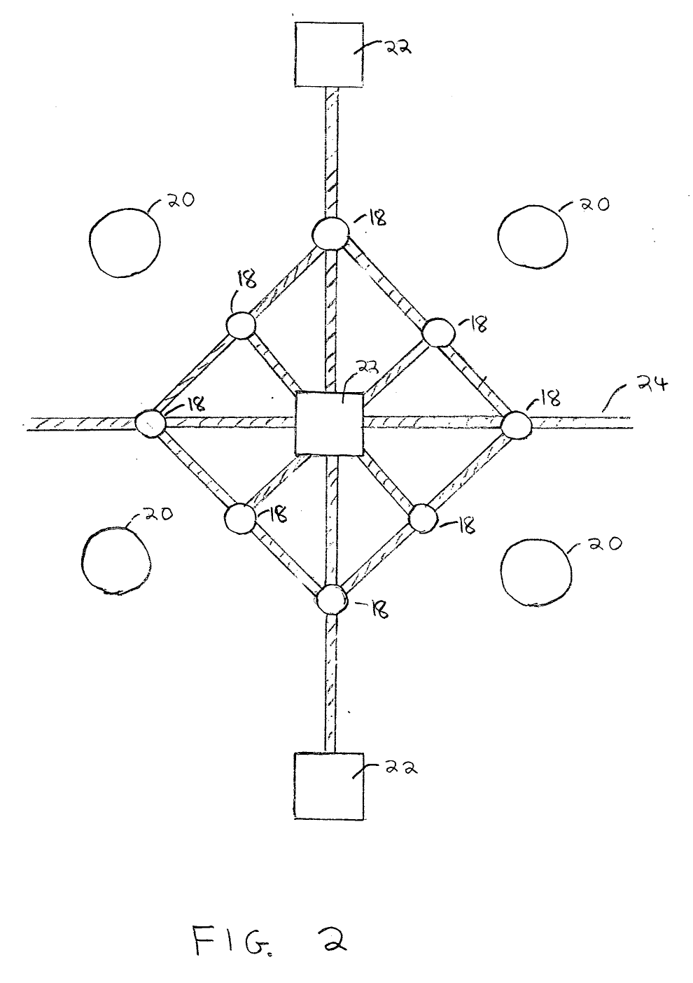

In the embodiment of the invention shown in FIG. 1 and FIG. 2, one or more holes (22) in the base allow for fluid to flow from the lower (14) to the upper side (16) of the base. The device may have one large hole with a plurality of puncturing elements (18) surrounding it or may have multiple holes with one or more puncturing elements (18) associated with each. The lower side (14) of the base further contains channels (24), which permit the interstitial fluid to move from the puncture sites to the holes (22) in the base. The lower side of the base further contains protrusions or “bumps” (20). These bumps are of a height sufficient to lift the base off the skin, so that the skin cannot conform around the bottom of the base and block the channels, but not so high as to prevent the puncturing elements (18) from penetrating at least the stratum comeum layer (100) of the skin and into the epidermal layer (102) to reach the interstitial fluid. Thus the bumps (20) will be of a length shorter than the puncturing elements (18). The cross section of the bumps may be similar to, narrower, or wider than the cross section of the puncturing elements. The bumps can range in dimensions from surface roughness (on the order of few microns in height and width), to features a few hundred microns wide and up to about 100 microns tall.

The bumps may be disposed on the comers or edges of the base, or additionally or alternatively in other locations on the base where they do not interfere with fluid flow to the holes. The bumps are depicted as having a rounded cross-section and convex tips; however, their shape may vary depending upon the processes used to produce them, and the type of puncturing elements used in the array. The bumps may have any shaped cross-section, such as rectangular, triangular, round, elliptical, etc., and may have tips that are flat, pointed, convex, or concave, preferably flat or convex. Illustrative examples of possible bump shapes are shown in FIG. 4.

The channels are depicted in FIG. 1 as having walls perpendicular to the base and a rectangular cross section; however, the channels may have walls which slope inwards or outwards with respect to the base, or walls which are curved, as depicted in FIG. 5.

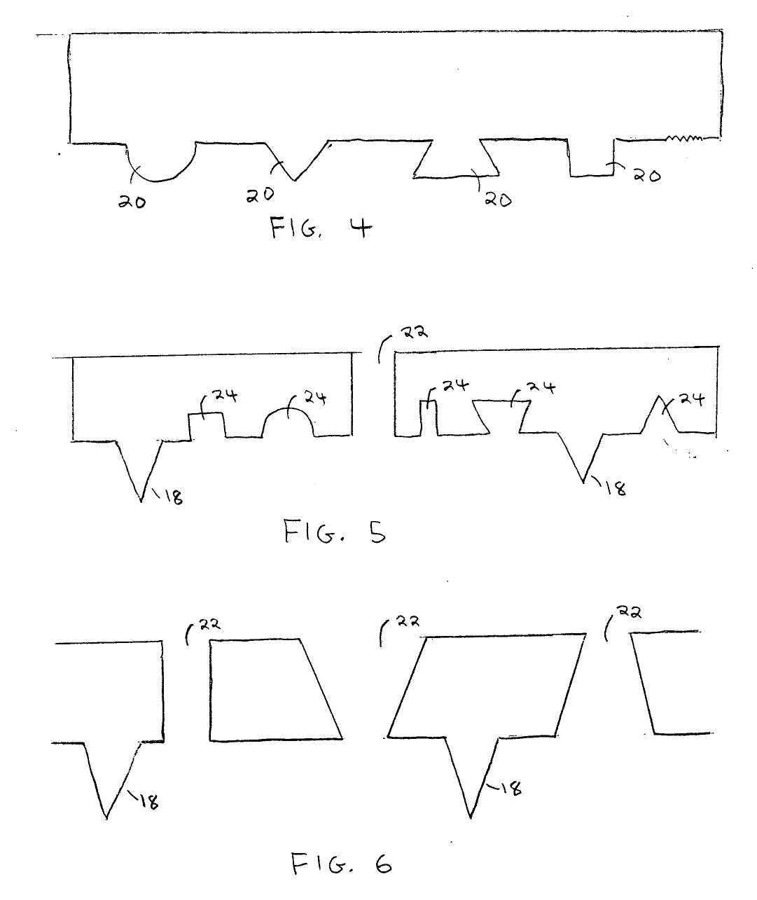

The holes are depicted in FIG. 2 as square, but may be of any shape, such as rectangular, triangular, round, elliptical, etc. The holes may have walls that are perpendicular to the base, or slanted at an angle, as shown in FIG. 6. The size of the holes may vary depending upon the material used to make the device, the fabrication processes, and the size and density of the puncturing elements. A preferred diameter range for the holes is from about 100 to about 500 microns

Alternative embodiments of the puncturing array (2) may be used with the collector of the invention. In an alternative embodiment, the puncturing elements are hollow microneedles, allowing fluid to flow from the lower to the upper side of the base without a need for openings, channels, or protrusions on the lower side of the base. Methods of making hollow microneedles are described, for example, in U.S. Pat. No. 6,663,820 and U.S. Pat. No. 6,503,231. In a further alternative, the puncturing elements are porous microneedles. Methods of making porous microneedles are described, for example, in U.S. Pat. No. 6,503,231. In a further alternative, the puncturing elements are microneedles or wedges with channels in their outer walls, as disclosed, for example, in WO 98/00193.

In the embodiment depicted in FIGS. 3H and 3I, the puncturing elements have outer walls with a roughened or textured surface so that pathways for fluid flow along the outer walls of the puncturing elements are created, allowing interstitial fluid to flow up to holes in the array base. In an alternative embodiment, the entire lower (skin contacting) surface of the array base may also have texture applied to it. A smooth surface tends to create larger adhesion forces than a rough one, and thus the application of texture would allow interstitial fluid to flow more smoothly. This is a technique that is used successfully in the hard disk drive industry to prevent the disk drive head from sticking to the media (disk), and fabrication processes for adding surface texture are well known in the art (see, for example, U.S. Pat. No. 5,079,657 and U.S. Pat. No. 6,683,754).

The transdermal agent sampling device of the invention may further comprise an agent sensing element (40), in contact with the upper side (16) of the array base. In the embodiment illustrated in FIG. 1, the sensing element comprises a first electrode (42), a chemical layer (46) for reacting with an agent in the interstitial fluid, with the chemical mixed in a mediating agent or bound in a matrix, and a second electrode (44). See, for example, U.S. Pat. No. 5,161,532, which is hereby expressly incorporated herein by reference. The electrodes are of porous material and permit the passage of interstitial fluid from one side through to the second side. The reaction of the chemical with the interstitial fluid produces an electrical signal which is picked up by the electrodes. The electrical signal can be measured by a detector (not shown). The detector is an amperometric detector which operates to detect the current generated by the electrodes.

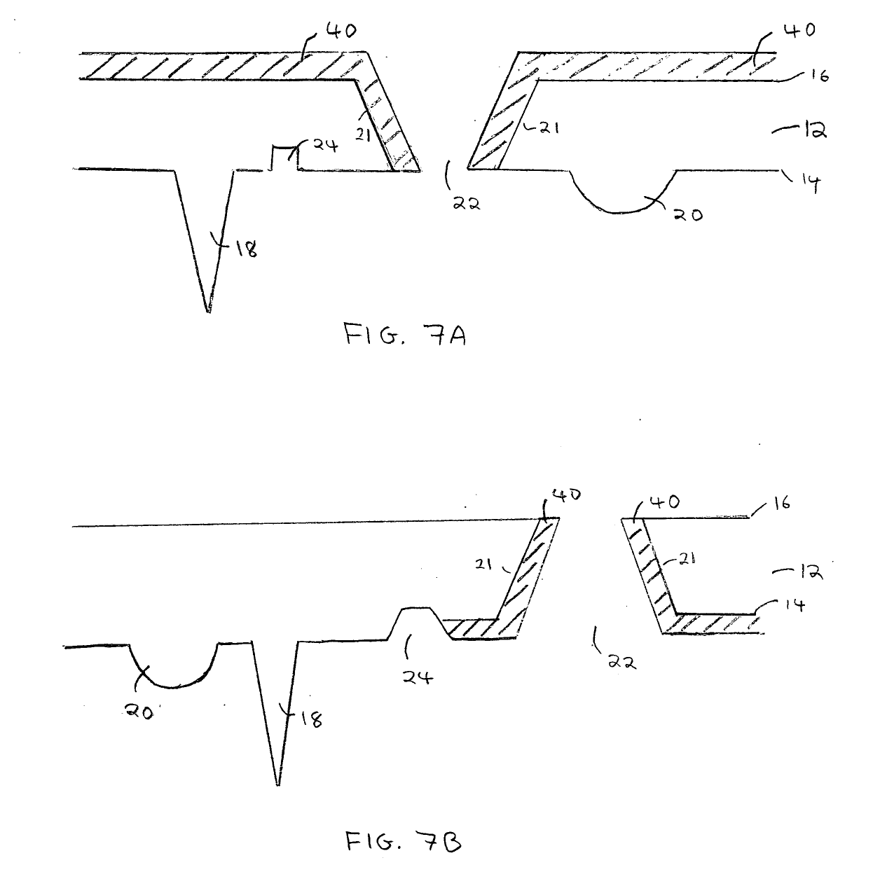

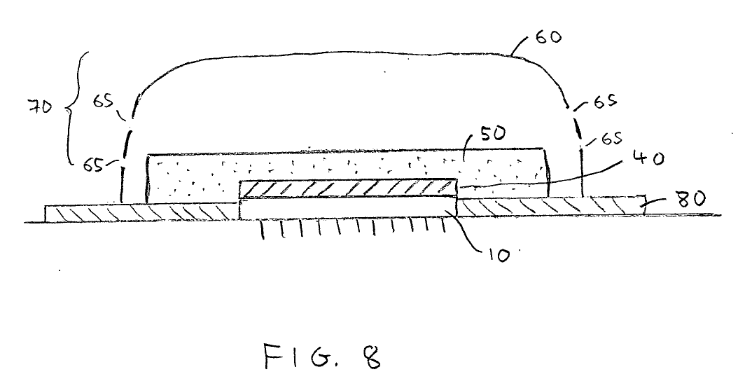

Other types of agent sensing elements may also be used, including but not limited to test strips which undergo a colorimetric change upon the detection of glucose or other agent, sensors which detect a pressure change upon the reaction of an agent with an enzyme in a hydrogel, or thermal chemical microsensors which detect heat released by the reaction of an agent with an enzyme. Enzyme-based sensors for the detection of various agents are well known in the art, and include, for example, glucose oxidase or glucose dehydrogenase, used to detect glucose. Sensing elements may also include antibodies specific to an agent as the assay material which interacts with the agent. The sensing elements may be porous, allowing fluid to flow through to the collector, or the holes in the base may extend through the sensing element as well, as depicted in FIGS. 7A and 7B.

The sensing element (40) need not be the same size as the base (12), and may be smaller in surface area. Depending on such factors as the chemistry involved in the sensor and the sensitivity of the measurement electronics, the sensor can be as small as 100 square microns in surface area. The total amount of fluid required for sampling may be as small as from about 0.2 to about 0.4 microliters.

In alternative embodiments of the invention, the sensing agent is incorporated into the puncturing elements. For example, an assay material such as glucose oxidase can be coated onto the external surface of hollow or solid puncturing elements, distributed within the pores of porous puncturing elements, or line or fill the bore(s) of hollow microneedles.

In further embodiments of the invention, the sensing agent (40) extends from the upper side (16) of the base along the walls (21) of the holes (22) to the lower side of the base (16), where it makes contact with the skin of a subject, as shown in FIG. 7A. In an alternative embodiment, the sensing agent (40) is disposed contiguous with at least a portion of the lower side (14) of the base, and extends along the walls (21) of the holes (22) to the upper side (16) of the base. These configurations of the sensor allow the extracted fluid to contact the sensing element more rapidly, allowing for more rapid sensing, and potentially for smaller sample sizes.

In one embodiment of the invention, a collector (70) for use with the skin piercing array (10) is shown in FIG. 8. The collector (70) comprises a large surface area membrane (50), which acts as a fluid reservoir and assists in drawing out the interstitial fluid by passive diffusion. The membrane (50) is disposed above and contiguously with the sensing element (40). The membrane (50) may also contact the base of the skin piercing array (10), in embodiments where the sensing element (40) is smaller in surface area than the array (10, and may further extend to contact the skin. In embodiments where the sensing agent is incorporated into the puncturing elements or disposed along the lower surface of the base, the membrane is disposed contiguously with the upper side (16) of the base.

Many natural and synthetic semi-permeable membranes are known in the art, including, for example, those disclosed in U.S. Pat. No. 4,077,407 and U.S. Pat. No. 4,014,334. Suitable membranes may be obtained from commercial sources including, for example, GE Osmonics Labstore (Minnetonka, MN). Suitable membranes from this source include, but are not limited to, OEM MAGNA PES (Polyethersulfone) membrane, OEM MAGNA nylon hydrophilic membrane, OEM PORETICS polycarbonate (PCTE) membrane, OEM PORETICS polyester (PETE) membrane, and OEM MAGNAPROBE nylon transfer membrane.

In one embodiment of the invention illustrated in FIG. 8, the device further comprises a housing (60). The housing preferably includes means for increasing evaporation of fluid from the device. In the embodiment shown in FIG. 8, the housing (60) contains slits (65) or openings which allow for the evaporation of interstitial fluid. Although shown as rectangular slits in the sides of the housing, these openings may be of any shape, and at alternate positions in the sides or top of the housing. In an alternative embodiment, the housing may contain a heating element, such as a thin heating strip. In either alternative, evaporation provides an increased driving force to suction out more fluid, helping to increase the fluid flow rate of the device. The slits are small enough to prevent fluids (water and sweat) from entering the device. Alternatively, the housing may be designed so that the slits can closed, so that the user may open them to the outside environment only when there is no likelihood of getting the device wet.

The housing may further contain electronic hardware and software for the detection and processing of the signal generated by the agent sensing element, and potentially for storage, transmission, processing and display of measured values, or for regulating the initiation of a sampling cycle. The housing may further comprise a mechanism for wireless or wire-based transmission of measured values to a remote device for analysis and/or display, such as an RF transmitter and/or receiver. The housing may further contain a power source, such as a thin film battery, for powering the electronics and, if incorporated, a heater, a micropump, or other components.

In certain embodiments, the devices of the invention may be made to adhere to the patient's body surface by various means, including an adhesive (80) applied to the lower (body-contacting) side of the device, or other anchoring elements on the array base of any of the embodiments discussed herein. The adhesive should have sufficient tack to insure that the array remains in place on the body surface during normal user activity, and yet permits reasonable removal after the predetermined wear period. In order for the device to be “user-friendly,” affixing the device to the skin should be relatively simple, and not require special skills. The patient can remove a peelaway backing to expose an adhesive coating, and then press the device onto a clean part of the skin, leaving it to monitor levels of an agent, such as glucose, for periods from 1 to 3 days.

The puncturing elements of the device, and the base to which the puncturing elements are attached or integrally formed, including any bumps, channels, or holes, can be constructed from a variety of materials, including metals, ceramics, semiconductors, organics, polymers, and composites. The puncturing elements must have the mechanical strength to remain intact and to collect biological fluid, while being inserted into the skin, while remaining in place for up to a number of days, and while being removed. The puncturing elements should preferably be sterilizable using standard methods.

The puncturing elements of the device can be constructed from a variety of materials, including metals and metal alloys, ceramics, semiconductors, organics, polymers, and composites. Preferred materials of construction include pharmaceutical grade stainless steel, titanium and titanium alloys consisting of nickel, molybdenum and chromium, metals plated with gold, platinum, and the like, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluorethylene (TEFLON(TM)), and polyesters.

The microneedle devices are made by microfabrication processes, by creating small mechanical structures in silicon, metal, polymer, and other materials. These microfabrication processes are based on well-established methods used to make integrated circuits and other microelectronic devices.

Microfabrication processes that may be used in making the puncturing elements include lithography; etching techniques, such as wet chemical, dry, and photoresist removal; thermal oxidation of silicon; electroplating and electroless plating; diffusion processes, such as boron, phosphorus, arsenic, and antimony diffusion; ion implantation; film deposition, such as evaporation (filament, electron beam, flash, and shadowing and step coverage), sputtering, chemical vapor deposition (CVD), epitaxy (vapor phase, liquid phase, and molecular beam), electroplating, screen printing, and lamination. See Madou M.J. “Fundamentals of microfabrication” CRC Press, Boca Raton (1997); Lau H.W. et al., Applied Physics Letters 67, 1877-79 (1995); and Zahn, J.D. et al, Biomedical Microdevices, Vol. 2, No. 4, 2000.

Alternatively, the arrays may be constructed of plastic or some other type of molded or cast material using a micromachining technique to fabricate the molds for a plastic microforming process (see, for example, U.S. Pat. 6,451,240 and U.S. Pat. 6,471,903).

As described above, the arrays are designed so as to prevent blockage of fluid flow by the conformation of skin around the puncturing elements. Thus there is no need to have a stiff array that avoids conforming to the local contours of the skin, and in fact a relatively flexible array may be preferred. This may be achieved by using an inherently flexible material, such as a flexible polymer or flexible metallic material, for at least the base of the device.

Additional Embodiments:

It is noted that the various aspects of the invention are not limited to use in combination. For example, the puncturing element arrays of the present invention are valuable for use in a range of applications. The puncturing element arrays of the invention can be used in conjunction with a wide variety of collector systems in addition to that disclosed in the Figures. The arrays of the present invention can be used with known sampling devices including, but not limited to, reverse iontophoresis, osmosis, passive diffusion, phonophoresis, and suction (i.e., negative pressure). Moreover, the collector of the invention may be used in conjunction with a wide variety of arrays in addition to that shown in the Figures, including, but not limited to those disclosed in U.S. Pat. No. 6,558,361, U.S. Pat. No. 6,652,478, WO 98/00193, U.S. Pat. No. 6,663,820, U.S. Pat. No. 6,503,231, U.S. Pat. No. 6,451,240, U.S. Pat. No. 6,471,903 and U.S. Pat. No. 6,219,574, all of which patents are hereby expressly incorporated by reference herein. The devices of the present invention may be used in combination with other techniques for further increasing transdermal flow rates, including but not limited to permeation enhancers, suction, electric fields, or ultrasound.

One of skill in the art will understand that further embodiments of the invention could include multianalyte sensors, in which agent sensing elements that detect different agents are disposed above distinct regions of the array base. Because the devices of the invention require only a small sample size, the surface area of each sensing element may be small, allowing a multianalyte sensor to be of a compact size.

The devices of the invention can also be used as components in a “smart patch” or regulation system, together with other elements including, but not limited to, electronics, power sources, transmitters, heaters, and pumps, as mentioned above. The devices of the invention might be used in combination with drug delivery means to provide a regulatory system that would, for example, withdraw fluid, calculate the concentration of glucose, determine the amount of insulin needed and deliver that amount of insulin.

Various features of the invention provide advantages for use in a long-term (e.g., 1-3 days) patch for agent sensing and monitoring. The devices of the invention require very little fluid sample for the sensor to measure the agent. Thus the array of puncturing elements can have a very small area, resulting in the disruption of a smaller skin area and therefore reduced skin irritation effects. Because the devices do not require large sample sizes, they permit more rapid and more frequent sampling. The devices of the invention do not require the use of electophoretic or ultrasound methods which can irritate the skin. The devices of the invention do not require large fluid reservoirs, allowing them to be compact. The compact and light devices of the invention place a minimal burden on an adhesive used to secure a device of the invention to a patient's skin, making them easier to use, and are less obtrusive and burdensome to the patient. The devices of the invention are designed to prevent blockage of fluid flow by the conformation of skin around the device; thus the devices can be made more flexible to contact the skin more effectively and be more comfortable to the user. The devices of the invention may be manufactured cheaply and easily using known microfabrication methods.

The description above should not be construed as limiting the scope of the invention, but as merely providing illustrations of some of the presently preferred embodiments of the invention.

We claim:

1. A device for transdermal agent sampling comprising:
(a) a base having a lower side and an upper side;
(b) a plurality of puncturing elements extending from the lower side of the base;
(c) a plurality of holes extending from the lower side of the base to the upper side of the base; and
(d) one or more protrusions extending from the lower side of the base, the protrusions of sufficient height to allow fluid to flow under the base while still permitting the puncturing members to penetrate through the stratum comeum of a subject.

2. The device of claim 1 further comprising a network of channels configured in the lower side of the base to interconnect the holes.

3. The device of claim 1 further comprising an agent sensing element contiguous with the upper side of the base.

4. The device of claim 2 wherein the agent sensing element is a glucose detector.

5. The device of claim 2 further comprising a collector.

6. A device for transdermal agent sampling comprising:
(a) a base having an upper side and a lower side, with a plurality of puncturing elements extending from the lower side of the base;
(b) an absorbent membrane contiguous with the upper side of the base; and
(c) means for increasing interstitial fluid evaporation.

7. The device of claim 6 wherein the means for increasing interstitial fluid evaporation is slits within a casing that houses said absorbent membrane.

8. The device of claim 6 wherein the means for increasing interstitial fluid evaporation is a heating element housed within said casing.

9. The device of claim 5 wherein the device further comprises a plurality of holes extending from the lower side of the base to the upper side of the base, a network of channels configured in the lower side of the base to interconnect the holes and one or more protrusions extending from the lower side of the base, the protrusions of sufficient height to allow fluid to flow under the base while still permitting the puncturing members to penetrate through the stratum corneum of a subject.

10. The device of claim 5 wherein the puncturing elements have textured outer walls.

11. The device of claim 5 wherein the lower side of the base and the outer walls of the puncturing elements have applied surface texture.

12. A method of transdermal monitoring of a selected analyte in a body comprising:
(a) Providing a device of claim 1; and
(b) contacting said device with the skin such that said plurality of puncturing elements puncture the skin to a depth sufficient to reduce the barrier properties thereof, resulting in a seepage of interstitial fluid from the skin through the holes in the base.

13. The method of claim 12 wherein the device further comprises an agent sensing element.

14. The method of claim 13 wherein the agent sensing element is a glucose detector.

15. The method of claim 12 further comprising treating the skin of a subject with one or more permeation enhancers prior to application of the device.

16. The method of claim 12 further comprising applying suction to enhance the rate of interstitial fluid flow.

17. The method of claim 12 wherein the device further comprises a collector.

18. The method of claim 17 wherein the collector comprises an absorbent membrane contiguous with the upper side of the base and means for increasing interstitial fluid evaporation.
